(19) United States
(12) Patent Application Publication
(10) Pub. No.: US 2025/0177770 A1
(43) Pub. Date: Jun. 5, 2025

(54) SUBSTANCE USE DISORDER PREVENTION OR TREATMENT WITH LOW INTENSITY AND HIGH FREQUENCY MAGNETIC STIMULATION

(71) Applicant: Actipulse Neuroscience Inc., Cambridge, MA (US)

(72) Inventors: Alain Andre CHÂTILLON, Mexico City (MX); Gabriel VILLAFUERTE, Mexico City (MX); Adrian CHÂTILLON, Mexico City (MX)

(21) Appl. No.: 18/836,939

(22) PCT Filed: Feb. 9, 2023
(86) PCT No.: PCT/US23/12683

§ 371 Date: Aug. 8, 2024

Related U.S. Application Data
(60) Provisional application No. 63/309,218, filed on Feb. 11, 2022.

(51) Int. Cl. A61N 2/00; A61N 2/02

(52) U.S. Cl. CPC A61N 2/006; A61N 2/02

(57) ABSTRACT

The present invention provides, in part, methods of treatment for substance use disorder comprising applying repetitive transcranial magnetic stimulation (rTMS) therapy to a patient in need thereof. The present invention also provides devices that generate a low intensity pulsed magnetic field and variable frequencies.

### CROSS-REFERENCE TO RELATED APPLICATION

This application claims the benefit of U.S. Provisional Patent Application No. 63/309,218, filed Feb. 11, 2022, the entire contents of which are herein incorporated by reference.

### FIELD OF THE INVENTION

The present invention provides, in part, clinical applications of a non-invasive brain stimulation, e.g., repetitive Transcranial Magnetic Stimulation (rTMS), as it pertains to neuromodulation in the treatment of substance abuse disorders.

### BACKGROUND

Among the main characteristics of a substance use disorder (SUD) are cognitive, behavioral and physiological symptoms, which are related to substance use despite associated problems (DSM-V, 2013). Overall, the diagnosis of a substance use disorder is based on a pathological pattern of behaviors related to use of the substance. The four main criteria are: impaired control, social impairment, risky use, and pharmacological criteria (DSM-V, 2013).

It is estimated that around 271 million people between 15 and 64 years old used drugs in 2017. This data increased by 30% as of 2019, compared to 2009. The data suggest that the increase is higher in the use of opioids in Africa, Asia, Europe and North America and the use of cannabis in North America, South America and Asia (UN, 2019).

It is estimated that 1 in 10 U.S. adults have a substance use disorder, which will involve the use of polysubstance. In 2014 in the United States 47,000 people died due to overdoses from multiple substance use. In this sense, it is important to develop treatments that consider an implementation of polysubstance use and not only focus on one. (McCabe, West, Jutkiewicz, & Boyd, C. 2017). There is a need to increase research on the comprehensive care of services that serve SUD, because by increasing primary care treatments in SUD opens the opportunity to reduce SUD related morbidity and mortality (Wu, McNeely, Subramaniam, Brady, Sharma, VanVeldhuisen, & Schwartz, 2017).

Transcranial magnetic stimulation (TMS) is a non-invasive, painless neurostimulation technique with a high safety profile that has been used as a neurologic and psychiatric treatment. However, the use of repetitive TMS as a treatment for neurocognitive diseases and disorders is expensive, requires a clinical setting, is hard to transport, is complex to use, requires external coolers, and may have severe adverse effects such as seizures. These disadvantages are the consequence of current TMS technology, which uses frequencies from 1 to 50 Hz and intense magnetic fields of around 10 million milligauss (i.e., around 1 Tesla).

There is a need for improved treatments for substance use disorder. There is also a need for devices and methods that employ a non-invasive brain stimulation, e.g. rTMS, without using large magnetic fields, and without focusing the magnetic waves at a certain point in the brain. There is a further need for a user-friendly device that is portable, appropriate for home use, and is safe to use without the need of a medical professional to apply it.

### SUMMARY OF THE INVENTION

Accordingly, the present invention provides low intensity and high frequency non-invasive brain stimulation, e.g., rTMS, methods to safely and effectively treat or prevent substance use disorder in a convenient manner, e.g., directly from home and without clinical supervision.

In one aspect, the present invention provides a non-invasive brain stimulation, e.g. rTMS, method for treating or preventing substance use disorder, comprising repetitively applying a magnetic pulse to the scalp of a patient in need thereof thereby stimulating neurons in the brain of the patient, wherein the magnetic pulse is applied (a) repetitively over the patient's brain (e.g., without limitation, the patient's left prefrontal dorsolateral cortex); and (b) at a frequency of about 10 to about 1000 Hz and an intensity of about 5,000 to about 200,000 milligauss (about 0.0005 Tesla to about 0.02 Tesla).

In various embodiments, the present invention contemplates applying a magnetic pulse using rTMS to a patient afflicted with substance use disorder. In various embodiments, the patient with substance use disorder presents as having one or more of the following symptoms: the individual consumes large amounts of substance for longer periods of time, may communicate a desire to decrease or regulate consumption, spends a lot of time consuming, seeking or recovering from the effects, craving, social, work, family and recreational activities are affected, substance use in risky situations, the individual continues consuming substances in spite of the physical or psychological problems they are causing, they present tolerance—need of higher doses to achieve the desired effect—and abstinence—it is produced when the concentrations of a substance decrease in an individual who has maintained a prolonged consumption. In various embodiments, the patient with substance use disorder presents as having one or more symptoms or criteria found in Diagnostic and Statistical Manual of Mental Disorders (DSM)-V, 2013.

In various embodiments, treating or preventing substance use disorder comprises diminishing impaired control over substance use; diminishing the time the time person spends obtaining, using or recovering from the substance and diminishing craving symptoms. In other embodiments, treating substance use disorder may comprise increasing the chance to consciously cease substance use by the person.

In various embodiments, treating or preventing substance use disorder comprises increasing the ability of a person to fulfill major social obligations such as, without limitation, going to work, school, etc. In other embodiments, treating or preventing substance use disorder may comprise diminishing withdrawal symptoms and diminishing tolerance from a drug or other substance.

In some embodiments, craving, withdrawal, dependence and other symptoms related to use disorder can be assessed by using one or more of the: The ASUS (Adult Substance Use Survey), the CAG, CAGE-AID modification, CRAFFT, the DAST (Drug Abuse Screening Test), DUSI-R (Drug Use Screening Inventory-Revised), MAST (Michigan Alcohol Screening Test), MAYSI-2 (Massachusetts Youth Screening Instrument—Version 2), PESQ (Personal Experience Screening Questionnaire), RAPS4 (Rapid Alcohol Problems Screen), SASSI-3 (Substance Abuse Subtle Screening Inventory, 3rd Edition), SASSI-A2 (Substance Abuse Subtle Screening Inventory—Adolescent, 2nd Edition), TAAD (Triage Assessment for Addictive Disorders), TICS (Two-Item Conjoint Screening Test), TWEAK, UNCOPE, e CAAPE (Comprehensive Addiction and Psychological Evaluation), CIDI V1.1 (Composite International Diagnostic Interview Version 1.1), GAIN-I (Global Appraisal of Individual Needs—Initial), SCID (Structured Clinical Interview for DSM), LOCI-2R (Level of Care Index-2 Revised), SUDDS-IV (Substance Use Disorder Diagnostic Interview-IV), CASI (Comprehensive Adolescent Severity Inventory), DISC-IV (Diagnostic Interview Schedule for Children—Version IV), GAIN-I (Global Appraisal of Individual Needs—Initial), GAIN-M90 (Global Appraisal of Individual Needs-M90), PADDI (Practical Adolescent Dual Diagnostic Interview), ASI (Addiction Severity Index), FAF (Family Assessment Form), GAIN-M90 (Global Appraisal of Individual Needs-M90), LOCI-2R (Level of Care Index-2 Revised), RAATE (Recovery Attitude and Treatment Evaluator), Risk Inventory for Substance Abuse—Affected Families, SDM (Structured Decision Making), Sacramento County Alcohol and Other Drug (AOD), Prototypes Stages of Change Form, Illinois Department of Children and Family Services Progress Matrix and Specialized Treatment and Recovery Services (STARS).

In various embodiments, treating or preventing substance use disorder comprises diminishing associated psychiatric symptoms such as depressive symptoms (e.g., anhedonia, apathy, insomnia, guilt, hopelessness), and/or anxiety symptoms and/or increasing quality of life in the patient in need thereof.

In various embodiments, treating or preventing substance use disorder comprises slowing memory loss or retaining or increasing memory capacity, memory function, or cognitive function in the patient in need thereof. In some embodiments, a method of slowing memory loss or retaining or increasing memory capacity, memory function, or cognitive function is provided, comprising administering rTMS described herein to a patient in need thereof.

In some embodiments, depressive symptoms, anxiety symptoms and quality of life can be assessed by using one or more of the Beck Depression Inventory, Hamilton Depression Rating Scale, Montgomery-Asberg Depression Rating Scale, Center for Epidemiologic Studies Depression Scale, Beck Hopelessness Scale, Geriatric Depression Scale, Cornell Dysthymia Rating Scale, Quick Inventory of Depressive Symptomatology, Reminiscence Functions Scale, MAST, Goldberg Anxiety and Depression Scale, Beck Anxiety Inventory, Hamilton Anxiety Rating Scale, Generalized Anxiety Disorder 7-item, Health Anxiety Inventory, The State-Trait Anxiety Inventory, Geriatric Anxiety Inventory, Taylor Manifest Anxiety Scale, Hospital Anxiety and Depression Scale, Anxiety Sensitivity Index, Athens Insomnia Scale, Insomnia Severity Index, Pittsburgh Sleep Quality Index, Sleep Quality Scale, Daytime Insomnia Symptom Scale, Insomnia Symptom Questionnaire, Pittsburgh Insomnia Rating Scale, Global Sleep Assessment Questionnaire, PROMIS Sleep Disturbance, Brief Fatigue Inventory, Epworth Sleepiness Scale, Functional Outcomes of Sleep Questionnaire, Mini Sleep Questionnaire, Bergen Insomnia Scale, Patient Health Questionnaire, Short Form Health Survey, Quality of Life Scale, Social Adjustment Scale—Self Report, McGill Quality of Life Questionnaire, Health-related quality of life, World Health Organization Quality of Life Instrument, Global Quality of Life Scale, Social Functioning Questionnaire, Life Satisfaction Index, Quality Of Life In Depression Scale, Assessment of Quality of Life, Control, Autonomy, Self-Realization and Pleasure (CASP-19), and Patient-Reported Outcomes Measurement Information System, amongst others. In embodiments, the present methods provide improvement in a patient or population of patients, as assessed by the aforementioned scales of this paragraph.

In some embodiments, memory capacity, memory function, cognitive function, or memory loss is assessed using one or more of the Montreal Cognitive Assessment (MoCA), Mini-mental state examination (MMSE), Verbal fluidity test, Frontal Assessment Battery (FAB), Geriatric Depression Scale (GDS-15), Clinical dementia rating (CDR), EuroQoL-5D, Daily Life Activities of Katz (ABVD), Lawton Daily Life Instrumental Activities Scale (AIVD), and Bayer Scale of Activities of Daily Living (B-ADL), Clinical Dementia Rating-Sum of Boxes (CDR-SB), and Repeatable Battery for the Assessment of Neuropsychological Status (RBANS). In embodiments, the present methods provide improvement in a patient or population of patients, as assessed by the aforementioned scales of this paragraph.

### DESCRIPTION OF THE FIGURES

FIG. 1A-B depicts the first part of a non-limiting example of the rTMS device. FIG. 1A shows the front view of the first part. FIG. 1B shows the back view of the first part.

FIG. 2 depicts the second part of a non-limiting example of the rTMS device.

FIG. 3 depicts a non-limiting example of the proposed use of the rTMS device. A shows the first part described in FIG. 1A-B and B depicts the headband described in FIG. 2.

FIG. 4 Depicts Changes in exhaled CO during the 8-week period of stimulation. There was a statistically significant difference in CO concentration in different time points: from baseline to week 3 (<0.01), week 5 (<0.01), week 6 (<0.01), and last week 7 (<0.01). CO, carbon monoxide; PPM, parts per million.

FIG. 5 depicts a non-limiting example of change in several scores before and after HFLI TMS. Changes in (a) BDI score, (b) BAI score, and (c) SF36% before and after HFLI TMS. BDI, Beck Depression Index; BAI, Beck Anxiety Index; SF36%, percentage of Short Form Health Survey; HFLI TMS, High Frequency and Low Intensity Transcranial Magnetic Stimulation.

### DETAILED DESCRIPTION OF THE INVENTION

In various aspects, the present invention provides, in part, methods for the treatment, prevention, or improvement of substance use disorder symptoms, including but not limited to, craving symptoms, withdrawal symptoms, tolerance symptoms and/or psychiatric symptoms associated to substance use disorder, included but not limited, to depressive symptoms, anxiety symptoms, quality of life and cognitive performance in patients with substance use disorder. In various aspects, the present invention provides a method using high frequency, low intensity transcranial magnetic stimulation and/or non-invasive brain stimulation using high frequency, low intensity magnetic fields.

### Repetitive Transcranial Magnetic Stimulation (rTMS)

In embodiments, the present invention provides methods for using non-invasive brain stimulation.

In various embodiments, the present invention provides for using transcranial magnetic stimulation (TMS) in a non-invasive and non-painful method to stimulate the cerebral cortex. In embodiments, the present invention contemplates a device that generates low-intensity pulsed magnetic fields and high frequencies. In some embodiments, rTMS activates or inhibits cortical activation by generating a magnetic field that, by Faraday's principle, generates an electrical field inside the brain. In some embodiments, the magnetic field is produced when an electric current passes through a coil. Further, in some embodiments, in order to generate an electric field inside the brain, the magnetic field starts and finishes rapidly. Without wishing to be bound by theory, if the magnetic field intensity stays constant too long, no electrical changes occur inside the brain. In addition, it is contemplated that when making use of magnetic fields of lower magnitude, neither a voltage transformer nor high currents are necessary. Indeed, the present invention contemplates a device that decreases potential adverse effects, facilitates its transport, and does not require external coolers for the operation.

In other embodiments, the present invention produces a magnetic field that not only activates or inhibits cortical neurons but also synchronizes cortical neural firing to the frequency of the magnetic pulses. This entrainment effect will change underlying oscillatory activity of neurons and, in consequence, modify substance use disorder.

In various embodiments, the present invention provides for using transcranial magnetic stimulation (TMS), wherein the magnetic pulse is applied at a frequency of about 10 to about 1000 Hz, about 10 to about 900 Hz, about 10 to about 800 Hz, about 10 to about 700 Hz, about 10 to about 600 Hz, about 10 to about 500 Hz, about 10 to about 400 Hz, about 10 to about 300 Hz, about 10 to about 200 Hz, about 100 to about 1000 Hz, about 300 to about 1000 Hz, about 400 to about 1000 Hz, about 500 to about 1000 Hz, about 600 to about 1000 Hz, about 700 to about 1000 Hz, about 800 to about 1000 Hz, or about 900 to about 1000 Hz. In specific embodiments, the magnetic pulse is applied at a frequency of about 10 Hz, about 20 Hz, about 30 Hz, about 40 Hz, about 50 Hz, about 60 Hz, about 70 Hz, about 80 Hz, about 90 Hz, about 100 Hz, about 200 Hz, about 300 Hz, about 400 Hz, about 500 Hz, about 600 Hz, about 700 Hz, about 800 Hz, about 900 Hz, or about 1000 Hz.

In embodiments, the present invention provides for using transcranial magnetic stimulation (TMS), wherein the magnetic pulse is applied at an intensity of about 1,000 to about 200,000 milligauss, or about 5,000 to about 150,000 milligauss, or about 7,000 to about 120,000 milligauss. In embodiments, the present invention provides for using transcranial magnetic stimulation (TMS), wherein the magnetic pulse is applied at an intensity of about 1,000 to about 200,000 milligauss, or about 1,000 to about 150,000 milligauss, or about 1,000 to about 100,000 milligauss, or about 1,000 to about 50,000 milligauss, or about 50,000 to about 150,000 milligauss, or about 10,000 to about 150,000 milligauss. In embodiments, the magnetic pulse is applied at an intensity of about 1,000 milligauss, about 2,000 milligauss, about 3,000 milligauss, about 4,000 milligauss, about 5,000 milligauss, about 6,000 milligauss, about 7,000 milligauss, about 8,000 milligauss, about 9,000 milligauss, about 10,000 milligauss, about 11,000 milligauss, about 12,000 milligauss, about 13,000 milligauss, about 14,000 milligauss, about 15,000 milligauss, about 16,000 milligauss, about 17,000 milligauss, about 18,000 milligauss, about 19,000 milligauss, about 20,000 milligauss, about 50,000 miligauss, about 100,000 miligauss, about 150,000 miligauss or 200,000 miligauss.

In embodiments, the present invention provides for using transcranial magnetic stimulation (TMS), wherein the magnetic pulse is applied at an intensity of about 0.0001 to about 0.002 Tesla, or about 0.0005 to about 0.0015 Tesla, or about 0.0007 to about 0.0012 Tesla. In embodiments, the magnetic pulse is applied at an intensity of about 0.0001 Tesla, about 0.0002 Tesla, about 0.0003 Tesla, about 0.0004 Tesla, about 0.0005 Tesla, about 0.0006 Tesla, about 0.0007 Tesla, about 0.0008 Tesla, about 0.0009 Tesla, about 0.001 Tesla, about 0.0011 Tesla, about 0.0012 Tesla, about 0.0013 Tesla, about 0.0014 Tesla, about 0.0015 Tesla, about 0.0016 Tesla, about 0.0017 Tesla, about 0.0018 Tesla, about 0.0019 Tesla, or about 0.002 Tesla.

In embodiments, the present invention provides for using transcranial magnetic stimulation (TMS), wherein the magnetic pulse generates an electric field of about 0.1 to about 10 V/m², about 0.5 to about 5 V/m², or about 0.5 to about 1.5 V/m². In embodiments, the magnetic pulse generates an electric field of about 1 V/m², about 2 V/m², about 3 V/m², about 4 V/m², about 5 V/m², about 6 V/m², about 7 V/m², about 8 V/m², about 9 V/m², about 10 V/m², about 20 V/m² or about 30 V/m²

In embodiments, the rTMS treatment method is undertaken once, or twice, or thrice, or four times daily. In particular embodiments, the method is undertaken once, twice, or thrice daily. In embodiments, the rTMS treatment method is undertaken for greater than about 10 minutes, about 15 minutes, about 20 minutes, about 25 minutes, about 30 minutes, about 35 minutes, about 40 minutes, about 45 minutes, about 50 minutes, about 55 minutes, or about 60 minutes. In some embodiments, the method is undertaken for about 15 to about 60 minutes. In particular embodiments, the method is undertaken for about 30 minutes. In various embodiments, the rTMS treatment method comprises magnetic pulses that are applied about 200 to about 1000 times per second, or about 200 to about 900 times per second, or about 200 to about 800 times per second, or about 200 to about 700 times per second, or about 200 to about 600 times per second, or about 200 to about 500 times per second, or about 200 to about 400 times per second, or about 200 to about 300 times per second, or about 300 to about 1000 times per second, or about 300 to about 900 times per second, or about 300 to about 800 times per second, or about 300 to about 700 times per second, or about 300 to about 600 times per second, or about 300 to about 500 times per second, or about 300 to about 400 times per second. In various embodiments, the rTMS treatment method comprises magnetic pulses that are applied about 300 to about 400 times per second. In certain embodiments, the pulses are applied about 10 times per second, about 20 times per second, about 30 times per second, applied about 40 times per second, about 50 times per second, about 80 times per second, about 300 times per second, about 310 times per second, about 320 times per second, about 330 times per second, about 340 times per second, about 350 times per second, about 360 times per second, about 370 times per second, about 380 times per second, about 390 times per second, or about 400 times per second. In various embodiments, the pulses last for about two seconds, about three seconds, about four seconds, or about five seconds. In certain embodiments, the pulses last for about four seconds, followed by one second without pulsing. In embodiments, a pulse is applied for a period of four seconds, followed by a one second pause in which no pulse is applied. In embodiments, the pulses are applied continuously during the whole time of the session without interruption.

In various embodiments, the rTMS treatment method contemplated by the present invention is applied chronically. For example, in some embodiments, the treatment is applied for greater than about 1 month, about 2 months, about 3 months, about 4 months, about 5 months, about 6 months, about 7 months, about 8 months, about 9 months, about 10 months, about 11 months, or about 1 year, or about 2 years, or about 3 years, or about 5 years, or about 10 years. In some embodiments, the treatment is applied for the life of the patient. In some embodiments, the treatment is self-applied.

In embodiments, the rTMS treatment method contemplated by the present invention is applied to the patient until symptoms, such as those described herein, improve or diminish. For example, the patient may undergo the treatment and experience a recovery of craving symptoms and hope to terminate the treatment. The patient may re-start the treatment if craving symptoms reemerges. Alternatively, the patient may undergo the treatment and experience a slowing or halting of craving symptoms, but not an improvement. In such a situation, in embodiments, the patient continues treatment chronically to mitigate or prevent further substance use disorder symptoms.

### rTMS Device

In some embodiments, the present invention provides devices and equipment for TMS, comprising an electromagnetic field generator and a coil that emits said electromagnetic field. In further embodiments, the magnetic field is produced when an electric current passes through a coil. In some embodiments, pulses of TMS are applied repeatedly with a frequency and a determined magnitude over a cortical area in order to modulate the cerebral cortex activity in the long term. In further embodiments, pulses of TMS are applied repeatedly at physiologically measured frequencies over a cortical area in order to synchronize the firing of neurons in the neocortex. In other embodiments, magnetic pulses are applied at higher frequencies (above 500 Hz) to inhibit the peripheral area of a cortical zone. In such embodiments, the method is rTMS.

It will be appreciated that the methods of the present invention are applied via a device capable of administering the transcranial magnetic stimulation. Indeed, in some embodiments, the TMS is applied using a device that is suitable for conducting electric current through a coil, thus generating a magnetic field. In embodiments, the device is suitable for home use and is optionally portable. In some embodiments, the device comprises a touch screen and optionally includes “digital health” applications and tele-health solutions. In some embodiments, the device is connected via Bluetooth to a smartphone.

In a non-limiting example, the present invention contemplates a device that generates a low intensity pulsed magnetic field and variable frequencies. In some embodiments, the device is a transcranial magnetic stimulation system able to generate pulsed magnetic fields in a frequency range from 10 to 1000 Hz. In further embodiments, the device is a transcranial magnetic stimulation system that generates a magnetic field with an intensity from 5,000 to 150,000 milligauss.

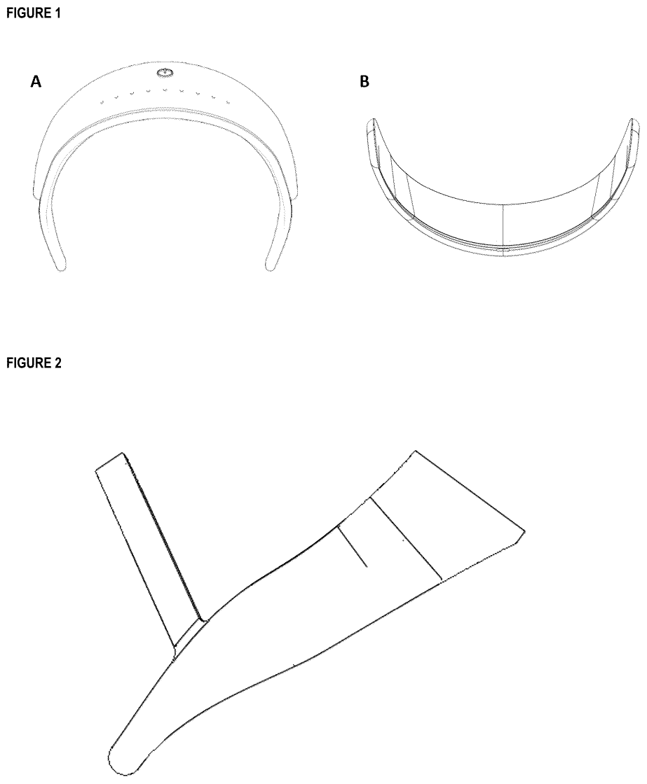

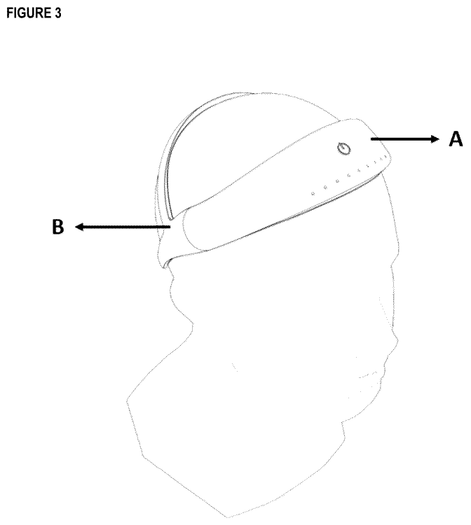

In an additional non-limiting embodiment, the TMS-generating device comprises three parts. In an embodiment, the first part of the device comprises an electronic unit and a coil that are located inside a plastic enclosure as depicted in FIG. 1A. In embodiments, the enclosure has two main faces which are described below. In certain embodiments, the front face is located an USB female connector where the device is charged; also in the front face, a series of light emitting diodes (LED) show the progress of the rTMS; also in the front face, a power button is located near the center of the enclosure. The back face comprises a smooth surface that adheres the plastic enclosure to a headband that allows to locate the plastic enclosure over the prefrontal cortex, as depicted in FIG. 1B. The electronics inside the plastic enclosure box consist of a PCB (Print Card Board), in which all the electronic components responsible to provide the electromagnetic waves to the coil by the connector are soldered, a coil and a battery.

In embodiments, the electronic circuit is composed of four parts: (1) a pulse generator from 10 to 1000 Hz, (2) an interruption generator, (3) control of light-emitting diodes (LEDs), (4) and a battery charging circuit. The electric current passes 10 to 1000 times per second through the coil and the flux is not interrupted during the duration of the stimulation session. In embodiments, the circuit is able to generate a pulsed magnetic field with an approximate intensity of 5,000 to 200,000 milligauss. In embodiments, the board has transistors, resistors, heat sinks, electrolytic capacitors, and diodes in order generate the pulse to the given frequency.

In embodiments, a light-emitting diode (LED) green is turned on to show the device is connected to the power adapter (5 v/2-3 Amp external power supply connected to a 110/220V socket). In an embodiment, a series of seven light-emitting diodes (LEDs) are horizontally placed in the part (B) (cover); these LEDs turn on serially to show that the device is working. In embodiments, a switch ON/OFF allows the user to start a treatment session.

In embodiments, the coil that transmits the magnetic pulse is located inside the plastic enclosure and over the left prefrontal dorsolateral cortex. In some embodiments, the device comprises a coil of 3-6 centimeters in diameter built from 50-100 turns of copper wire 99.5% pure, gauge 24-26 AWG. In further embodiments, a double coverage of insulating material covers the coil winding.

In embodiments, a battery or batteries are located inside the plastic enclosure and are used as a power source. The power source supplies the electronics and coil with the necessary current to operate the device and generate the magnetic pulses intended to be applied over the skull.

In embodiments, the second part which comprises the device is a headband that allows to place the plastic enclosure in the correct position over the skull, as depicted in FIG. 2. In a non-limiting embodiment, this headband can be fabricated from plastic, steel, aluminum or other materials. In other embodiments, the headband can have fabric in the side that fits the skull in order to comfortably fit the subject's head.

Finally, in a non-limiting embodiment, the third part of the device comprises a power adapter. In certain embodiments, the power adapter provides an alternate current to direct current transformer, and its input is from 120 to 240 Volts with a frequency of 60 Hz, and its output is from 5V and 1-3 Amperes.

### Methods of Treatment

In aspects, the present invention contemplates methods for slowing or preventing a conversion of recreational or pharmaceutical drug use to substance use disorder. In aspects, the present invention contemplates methods for treating or preventing substance use disorder using non-invasive brain stimulation, e.g. rTMS. In various embodiments, the method of rTMS comprises repetitively applying a magnetic pulse to the scalp of a patient in need thereof, thereby stimulating neurons in the brain of the patient, wherein the magnetic pulse is applied (a) repetitively over the patient's brain (e.g., without limitation, the patient's left prefrontal dorsolateral cortex); and (b) at a frequency of about 100 to about 1000 Hz and an intensity of about 5,000 to about 150,000 milligauss. In various embodiments, methods of the present invention are suitable for home use and can be performed without the need for a medical professional.

In aspects, the present invention contemplates methods for slowing or preventing a conversion of recreational or pharmaceutical drug use to opioid use disorder. In aspects, the present invention contemplates methods for treating or preventing opioid use disorder using non-invasive brain stimulation, e.g. rTMS. In various embodiments, the method of rTMS comprises repetitively applying a magnetic pulse to the scalp of a patient in need thereof, thereby stimulating neurons in the brain of the patient, wherein the magnetic pulse is applied (a) repetitively over the patient's brain (e.g., without limitation, the patient's left prefrontal dorsolateral cortex); and (b) at a frequency of about 100 to about 1000 Hz and an intensity of about 5,000 to about 150,000 milligauss. In various embodiments, methods of the present invention are suitable for home use and can be performed without the need for a medical professional.

In various embodiments, rTMS methods of the present invention stimulate neurons around about 1 to about 4 cm from the skull, about 2 to about 4 cm from the skull, about 1 to about 3 cm from the skull, or about 2 to about 3 cm from the skull. In some embodiments, the method stimulates neurons throughout the patient's brain. In further embodiments, the method substantially stimulates neurons in the left hemisphere of the patient's brain. In still further embodiments, the method substantially stimulates neurons in the frontal lobe of the patient's brain. In embodiments, the method substantially stimulates neurons in the cerebral cortex of the patient. In embodiments, the method stimulates neurons outside of the patient's left prefrontal dorsolateral cortex.

In various embodiments, the present invention provides for rTMS treatment methods that prevent or delay the progression of recreational or pharmaceutical drug use to substance use disorder. In some embodiments, the treatment method improves and/or prevents craving symptoms and traits of the patient. In some embodiments, the treatment method improves other symptoms related to substance use disorder. Further embodiments of the present invention include methods for treating psychiatric symptoms linked to substance use disorder, including, but not limited to, depression, anxiety, and cognitive decline.

In various embodiments, the present invention includes the treatment or prevention of substance use disorder, optionally which comprises reduction in length of a depressive episode. In various embodiments, the preventing or treating comprises recovery or improving of an anti-craving effect of the patient's pre-existent anti-substance use treatment regimen. In still other various embodiments, the preventing or treating comprises a reduction in the rate of relapse after successful substance use disorder. In further various embodiments, the present method comprises prevention or reversal of loss of efficacy of the patient's pre-existent treatment, which may, for example, cause one or more of a reduction in side effects and an increase in patient adherence.

In some embodiments, the treatment method reduces or abrogates addiction to one or more of alcohol, caffeine, cannabis, hallucinogens, inhalants, opioids, sedatives, hypnotics, anxiolytics, stimulants, cocaine, MDMA, and/or tobacco. In some embodiments, the treatment method improves and/or prevents craving for one or more of alcohol, caffeine, cannabis, hallucinogens, inhalants, opioids, sedatives, hypnotics, anxiolytics, stimulants, cocaine, MDMA, and/or tobacco.

In embodiments, the treatment method reduces or abrogates addiction to nicotine and/or tobacco. In some embodiments, the treatment method improves and/or prevents craving for nicotine and/or tobacco. In some embodiments, the treatment method improve cause smoking cessation or a reduction in smoking frequency. In some embodiments, the treatment method reduces or abrogates addiction to one or more opioids. In some embodiments, the treatment method improves and/or prevents craving for opioids.

In embodiments, the treatment method curtails or prevents exacerbation of SUD

### Patient Selection

In various embodiments, the present invention provides treatment of a patient that is afflicted with substance use disorder, including substance use disorder caused by several substances including, but not limited, to alcohol, caffeine, cannabis, hallucinogens, inhalants, opioids, sedatives, hypnotics, anxiolytics, stimulants, cocaine, MDMA, and/or tobacco.

In various embodiments, the present invention provides treatment of a patient that is afflicted with substance use disorder, including substance specific induced disorders such as intoxication, withdrawal or substance induced mental disorders. In various embodiments, the present invention provides treatment for withdrawal caused by alcohol, caffeine, cannabis, hallucinogens, inhalants, opioids, sedatives, hypnotics, anxiolytics, stimulants, cocaine, MDMA, and/or tobacco. In various embodiments, the present invention provides treatment for intoxication caused by alcohol, caffeine, cannabis, hallucinogens, inhalants, opioids, sedatives, hypnotics, anxiolytics, stimulants, cocaine, MDMA, and/or tobacco. In various embodiments, the present invention provides treatment for substance induced mental disorders caused by alcohol, caffeine, cannabis, hallucinogens, inhalants, opioids, sedatives, hypnotics, anxiolytics, stimulants, cocaine, MDMA, and/or tobacco.

In some embodiments, the patient afflicted with SUD presents as having the diagnostic criteria specified by the 5th edition Diagnostic and Statistical Manual of Mental Disorders.

In some embodiments, the present invention relates to a method of treating SUD, including by way of non-limiting example, breakthrough SUD and/or treatment-refractory SUD.

In further embodiments, the patient presents as having a test score of one or more identified instruments for assessment of SUD symptoms that is considered to indicate the presence of SUD. For example, in embodiments, the patient presents as having a Drug Abuse Screening Test (DAST) score of 12 or more points; or CAGE-AID of 2 or more.

The efficacy of treating SUD using methods and compositions of the present invention may be assessed by various methods. For example, Addiction Severity Index (ASI) works as a research instrument for program evaluation to determine the extent to which addiction treatment programs achieved improvements across seven areas: alcohol use, drug use, psychiatric status, employment status, medical status, legal status, and family/social relationships. The Recovery Attitude and Treatment Evaluator (RAATE) evaluates the acceptance/resistance to treatment, acceptance/resistance to continuing recovery efforts, acuity of medical conditions, acuity of psychiatric conditions, and the recovery environment.

In further embodiments, the patient presents as having a test score of two or more identified instruments for assessment of SUM symptoms and behaviors that is considered to indicate problems with the use of substances, such tests being found in Table 1 herein.

TABLE 1
Instruments for assessment of symptoms related to SUD

| Aspects of SUD | Abbreviation | Full name |
|---|---|---|
| Screening Tools | ASUS | Adult Substance Use Survey |
| | AUDIT | Alcohol Use Disorders |
| | | Identification Test |
| | CAGE | CAGE |
| | CAGE-AID | CAGE-AID MODIFICATION |
| | MODIFICATION | |
| | CRAFFT | ** |
| | DAST | Drug Abuse Screening Test |
| | DUSI-R | Drug Use Screening |
| | | Inventory-Revised |
| | MAST | Michigan Alcohol Screening Test |
| | MAYSI-2 | Massachusetts Youth Screening |
| | | Instrument-Version 2 |
| | PESQ | Personal Experience Screening |
| | | Questionnaire |
| | RAPS4 | Rapid Alcohol Problems Screen |
| | SASSI-3 | Substance Abuse Subtle Screening |
| | | Inventory, 3rd Edition |
| | SASSI-A2 | Substance Abuse Subtle Screening |
| | | Inventory-Adolescent, 2nd Edition |
| | TAAD | Triage Assessment for |
| | | Addictive Disorders |
| | TICS | Two-Item Conjoint Screening Test |
| | TWEAK | ** |
| | UNCOPE | ** |
| Assessment | CAAPE | Comprehensive Addiction and |
| Tools for SUD | | Psychological Evaluation |
| | CIDI V1.1 | Composite International Diagnostic |
| | | Interview Version 1.1 |
| | GAIN-I | Global Appraisal of Individual |
| | | Needs-Initial |
| | SCID | Structured Clinical Interview |
| | | for DSM |
| Treatment | LOCI-2R | Level of Care Index-2 Revised |
| planning and | | |
| placement | | |
| Instruments for | SUDDS-IV | Substance Use Disorder |
| Adolescents | | Diagnostic Interview-IV |
| | CASI | Comprehensive Adolescent |
| | | Severity Inventory |
| | DISC-IV | Diagnostic Interview Schedule |
| | | for Children-Version IV |
| | GAIN-I | Global Appraisal of Individual |
| | | Needs-Initial For adolescents |
| | GAIN-M90 | Global Appraisal of Individual |
| | | Needs-M90 |
| | PADDI | Practical Adolescent Dual |
| | | Diagnostic Interview |
| Planning and | ASI | Addiction Severity Index |
| Monitoring Tools | | |
| Regarding SUD | | |
| | FAF | Family Assessment Form |
| | GAIN-M90 | Global Appraisal of |
| | | Individual Needs-M90 |
| | LOCI-2R | Level of Care Index-2 Revised |
| | RAATE | Recovery Attitude and |
| | | Treatment Evaluator |
| | ** | Risk Inventory for Substance |
| | | Abuse-Affected Families |
| | SDM | Structured Decision Making |
| Instruments | AOD | Sacramento County Alcohol |
| Used by States | | and Other Drug |
| and Treatment | | |
| Providers | | |
| | ** | Prototypes Stages of |
| | | Change Form |
| | ** | Illinois Department of Children |
| | | and Family Services Progress |
| | | Matrix |
| | STARS | Specialized Treatment and |
| | | Recovery Services |
| Depressive | BDI | Beck Depression Inventory |
| symptoms | | |
| | HDRS | Hamilton Depression |
| | | Rating Scale |
| | MADRS | Montgomery-Asberg Depression |
| | | Rating Scale |
| | CES-D | Center for Epidemiologic |
| | | Studies Depression Scale |
| | GDS | Geriatric Depression Scale |
| | QIDS | Quick Inventory of Depressive |
| | | Symptomatology |
| | RFS | Reminiscence Functions Scale |
| | CDRS | Cornell Dysthymia Rating Scale |
| | BHS | Beck Hopelessness Scale |
| | Zung SDS | Zung Self-Rating Depression Scale |
| | PROMIS-D | PROMIS Depression Subscale |
| Anxiety | BAI | Beck Anxiety Inventory |
| Symptoms | | |
| | HARS | Hamilton Anxiety Rating Scale |
| | HAI | Health Anxiety Inventory |
| | STAI | State-Trait Anxiety Inventory |
| | GAI | Geriatric Anxiety Inventory |
| | ASI | Anxiety Sensitivity Index |
| | TMAS | Taylor Manifest Anxiety Scale |
| Anxiety and | GADS | Goldberg Anxiety and |
| Depressive | | Depression Scale |
| Symptoms | | |
| | HADS | Hospital Anxiety and |
| | | Depression Scale |
| Sleep | AIS | Athens Insomnia Scale |
| Symptoms | | |
| | ISI | Insomnia Severity Index |
| | PSQI | Pittsburgh Sleep Quality Index |
| | SQS | Sleep Quality Scale |
| | DISS | Daytime Insomnia Symptom Scale |
| | ISQ | Insomnia Symptom Questionnaire |
| | PIRS | Pittsburgh Insomnia Rating Scale |
| | GSAQ | Global Sleep Assessment |
| | | Questionnaire |
| | PROMIS-SD | PROMIS Sleep Disturbance |
| | | subscale |
| | BFI | Brief Fatigue Inventory |
| | ESS | Epworth Sleepiness Scale |
| | FOSQ | Functional Outcomes of |
| | | Sleep Questionnaire |
| | MSQ | Mini Sleep Questionnaire |
| | BIS | Bergen Insomnia Scale |
| Cognitive | MMSE | Mini-Mental State Examination |
| Impairment | | |
| | MoCA | Montreal Cognitive Assessment |
| | CAMCOG | Cambridge Cognitive Examination |
| | Mini-Cog | Mini-Cog |
| | CDT | Clock Drawing Test |
| | COGNISTAT | Neurobehavioral Cognitive |
| | | Status Examination |
| | ACE | Addenbrooke's Cognitive |
| | | Examination |
| | WAIS-R | Wechsler Adult Intelligence |
| | | Scale-Revised |
| | Qmci | Quick Mild Cognitive |
| | | Impairment Screen |
| | NTB | Neuropsychological |
| | | Test Battery |
| | GPCOG | General Practitioner |
| | | Assessment of Cognition |
| Quality | PHQ-9 | Patient Health Questionnaire |
| of Life | | |
| | SF-36 | Short Form Health Survey |
| | QOLS | Quality of Life Scale |
| | SAS-SR | Social Adjustment |
| | | Scale-Self Report |
| | MQOL | McGill Quality of Life |
| | | Questionnaire |
| | HRQoL | Health-Related Quality |
| | | of Life |
| | WHOQOL- | World Health Organization |
| | BREF | Quality of Life Instrument |
| | GQOL | Global Quality of Life Scale |
| | SFQ | Social Functioning |
| | | Questionnaire |
| | LSI | Life Satisfaction Index |
| | QLDS | Quality of Life in |
| | | Depression Scale |
| | AQoL | Assessment of Quality |
| | | of Life |
| | EQ-5D | EQ-5D |
| | CASP-19 | Control, Autonomy, Self- |
| | | Realization and Pleasure |
| | PROMIS | Patient-Reported Outcomes |
| | | Measurement Information System |

In some embodiments, a patient that has been treated with rTMS described herein shows an improvement (i.e., an increase or a decrease) in the patient's score of one or more instruments described in Table 1 as compared to baseline (e.g., prior to treatment or at an earlier time point during treatment). In some embodiments, the patient's score of one or more instruments described in Table 1 increases or decreases by at least 5%, or by at least 10%, or by at least 15%, or by at least 20%, or by at least 25%, or by at least 30%, or by at least 35%, or by at least 40%, or by at least 45%, or by at least 50%, or by at least 55%, or by at least 60%, or by at least 65%, or by at least 70%, or by at least 75%, or by at least 80%, or by at least 85%, or by at least 90%, or by at least 95%. In some embodiments, a patient that has been treated with rTMS described herein shows a slowing of the rate of increase or decrease in their score of one or more instruments described in Table 1 by at least 5%, or by at least 10%, or by at least 15%, or by at least 20%, or by at least 25%, or by at least 30%, or by at least 35%, or by at least 40%, or by at least 45%, or by at least 50%, or by at least 55%, or by at least 60%, or by at least 65%, or by at least 70%, or by at least 75%, or by at least 80%, or by at least 85%, or by at least 90%, or by at least 95%. In some embodiments, a stable score of one or more instruments described in Table 1 compared to baseline (e.g., prior to treatment or at an earlier time point during treatment) may be indicative of improvement, slowing, delay, or cessation of SUD, or a lack of appearance of new clinical, functional, or SUD symptoms or impairments, or an overall stabilization of disease activity.

In some embodiments, efficacy of treatment on SUD is assessed by The Recovery Attitude and Treatment Evaluator (RAATE). In an embodiment, efficacy may be demonstrated by a reduction in RAATE score of at least about 50%, about 55%, about 60%, about 65%, about 70%, about 75%, about 80%, about 85%, about 90%, about 95%, or about 100%, which signify improved response to treatment. Alternatively, efficacy may be demonstrated by a RAATE score of less than about 15, about 14, about 13, about 12, about 11, about 10, about 9, about 8, about 7.5, about 7, about 6.5, about 6, about 5.5, about 5, about 4.5, about 4, about 3.5, about 3, about 2.5, about 2, about 1.5, about 1, which signify increased remission following treatment. In some embodiments, the pre-treatment patient has a RAATE score of greater than about 23, or between about 19-23, or between about 14-18, or between about 8-13. In some embodiments. In some embodiments, the RAATE score effect size between patients not receiving low dose rTMS and patients receiving rTMS is about 30, or about 25, or about 20, or about 15, or about 14, or about 13, or about 12, or about 11, or about 10, or about 9, or about 8, or about 7, or about 6, or about 5. In some embodiments, the RAATE score effect size between patients not receiving low dose rTMS and patients receiving rTMS is between about 5 to about 15 or about 5 to about 10. In some embodiments, the rTMS causes any of the above reductions in RAATE score within about 7 days, or about 10 days, or about 14 days from initiation of treatment with rTMS. In some embodiments, any of the above reductions in RAATE score mediated by the rTMS is durably maintained in the patient (e.g. for about, or greater than about, 3 weeks, or about, or greater than about, 1 month, or about, or greater than about, 2 months, or about, or greater than about, 3 months, or about, or greater than about, 6 months, or about, or greater than about, 1 year). In an embodiment, efficacy may be demonstrated by a reduction in Structured Decision Making (SDM) score of at least about 50%, about 55%, about 60%, about 65%, about 70%, about 75%, about 80%, about 85%, about 90%, about 95%, or about 100%, which signify improved response to treatment. Alternatively, efficacy may be demonstrated by a SDM score of less than about 15, about 14, about 13, about 12, about 11, about 10, about 9, about 8, about 7.5, about 7, about 6.5, about 6, about 5.5, about 5, about 4.5, about 4, about 3.5, about 3, about 2.5, about 2, about 1.5, about 1, which signify increased remission following treatment. In some embodiments, the pre-treatment patient has a SDM score of greater than about 23, or between about 19-23, or between about 14-18, or between about 8-13. In some embodiments. In some embodiments, the SDM score effect size between patients not receiving rTMS and patients receiving rTMS is about 30, or about 25, or about 20, or about 15, or about 14, or about 13, or about 12, or about 11, or about 10, or about 9, or about 8, or about 7, or about 6, or about 5. In some embodiments, the SDM score effect size between patients not receiving rTMS and patients receiving rTMS is between about 5 to about 15 or about 5 to about 10. In some embodiments, the rTMS causes any of the above reductions in SDM score within about 7 days, or about 10 days, or about 14 days from initiation of treatment with rTMS. In some embodiments, any of the above reductions in SDM score mediated by the rTMS is durably maintained in the patient (e.g. for about, or greater than about, 3 weeks, or about, or greater than about, 1 month, or about, or greater than about, 2 months, or about, or greater than about, 3 months, or about, or greater than about, 6 months, or about, or greater than about, 1 year).

In some embodiments, efficacy of treatment on depression is assessed by the CAGE (or CAGE-AID MODIFICATION). In an embodiment, efficacy may be demonstrated by a reduction in CAGE score of at least about 50%, about 55%, about 60%, about 65%, about 70%, about 75%, about 80%, about 85%, about 90%, about 95%, or about 100%, which signify improved response to treatment. Alternatively, efficacy may be demonstrated by a CAGE score of less than about 15, about 14, about 13, about 12, about 11, about 10, about 9, about 8, about 7.5, about 7, about 6.5, about 6, about 5.5, about 5, about 4.5, about 4, about 3.5, about 3, about 2.5, about 2, about 1.5, about 1, which signify increased remission following treatment. In some embodiments, the pre-treatment patient has a CAGE score of greater than about 34, or between about 20-34, or between about 7-19. In some embodiments. In some embodiments, the CAGE score effect size between patients not receiving rTMS and patients receiving rTMS is about 40, or about 35, or about 30, or about 25, or about 20, or about 15, or about 14, or about 13, or about 12, or about 11, or about 10, or about 9, or about 8, or about 7, or about 6, or about 5. In some embodiments, the CAGE score effect size between patients not receiving rTMS and patients receiving rTMS is between about 5 to about 15 or about 5 to about 10. In some embodiments, the rTMS causes any of the above reductions in CAGE score within about 7 days, or about 10 days, or about 14 days from initiation of treatment with rTMS. In some embodiments, any of the above reductions in CAGE score mediated by the rTMS is durably maintained in the patient (e.g. for about, or greater than about, 3 weeks, or about, or greater than about, 1 month, or about, or greater than about, 2 months, or about, or greater than about, 3 months, or about, or greater than about, 6 months, or about, or greater than about, 1 year).

In an embodiment, efficacy may be demonstrated by a reduction in CAGE-AID MODIFICATION score of at least about 50%, about 55%, about 60%, about 65%, about 70%, about 75%, about 80%, about 85%, about 90%, about 95%, or about 100%, which signify improved response to treatment. Alternatively, efficacy may be demonstrated by a CAGE-AID MODIFICATION score of less than about 15, about 14, about 13, about 12, about 11, about 10, about 9, about 8, about 7.5, about 7, about 6.5, about 6, about 5.5, about 5, about 4.5, about 4, about 3.5, about 3, about 2.5, about 2, about 1.5, about 1, which signify increased remission following treatment. In some embodiments, the pre-treatment patient has a CAGE-AID MODIFICATION score of greater than about 34, or between about 20-34, or between about 7-19. In some embodiments. In some embodiments, the CAGE-AID MODIFICATION score effect size between patients not receiving rTMS and patients receiving rTMS is about 40, or about 35, or about 30, or about 25, or about 20, or about 15, or about 14, or about 13, or about 12, or about 11, or about 10, or about 9, or about 8, or about 7, or about 6, or about 5. In some embodiments, the CAGE-AID MODIFICATION score effect size between patients not receiving rTMS and patients receiving rTMS is between about 5 to about 15 or about 5 to about 10. In some embodiments, the rTMS causes any of the above reductions in CAGE-AID MODIFICATION score within about 7 days, or about 10 days, or about 14 days from initiation of treatment with rTMS. In some embodiments, any of the above reductions in CAGE-AID MODIFICATION score mediated by the rTMS is durably maintained in the patient (e.g. for about, or greater than about, 3 weeks, or about, or greater than about, 1 month, or about, or greater than about, 2 months, or about, or greater than about, 3 months, or about, or greater than about, 6 months, or about, or greater than about, 1 year).

In some embodiments, efficacy of treatment on SUD is assessed by the Drug Abuse Screening Test (DAST). In an embodiment, efficacy may be demonstrated by a reduction in DAST score to about 4, about 3.5, about 3, about 2.5, about 2, about 1.5, about 1, or about 0.5, which signify clinical response. In an embodiment, efficacy may be demonstrated by a reduction in CGI-I score to about 4, about 3.5, about 3, about 2.5, about 2, about 1.5, about 1, or about 0.5, which signify clinical response and improvement. In some embodiments, the pre-treatment patient has a DAST score effect size between patients not receiving rTMS and patients receiving rTMS is about 1.5, or about 1.3, or about 1.0, or about 0.7, or about 0.5. In some embodiments, the rTMS causes any of the above reductions in DAST score within about 7 days, or about 10 days, or about 14 days from initiation of treatment with rTMS. In some embodiments, any of the above reductions in DAST score mediated by the rTMS is durably maintained in the patient (e.g. for about, or greater than about, 3 weeks, or about, or greater than about, 1 month, or about, or greater than about, 2 months, or about, or greater than about, 3 months, or about, or greater than about, 6 months, or about, or greater than about, 1 year).

In various embodiments, The Michigan Alcohol Screening Test (MAST) is a 25-item screen developed in 1971 and with the CAGE has been one of the most widely used to screen for diagnosable abuse or dependence. Focuses on alcohol only and therefore must be paired with an instrument like the DAST that screens for drug disorders. It is long for a screening instrument. It screens for—alcoholism, Il a non-diagnostic term, and is not based on the diagnostic criteria of the DSM-IV.

In some embodiments, a patient that has been treated with rTMS described herein shows an improvement (i.e., a decrease) in the patient's MAST five-digit score compared to baseline (e.g., prior to treatment or at an earlier time point during treatment). In some embodiments, the patient's MAST five-digit score decreases by at least 5%, or by at least 10%, or by at least 15%, or by at least 20%, or by at least 25%, or by at least 30%, or by at least 35%, or by at least 40%, or by at least 45%, or by at least 50%. In some embodiments, a patient that has been treated with rTMS described herein shows a slowing of the rate of increase in their MAST five-digit score by at least 5%, or by at least 10%, or by at least 15%, or by at least 20%, or by at least 25%, or by at least 30%, or by at least 35%, or by at least 40%, or by at least 45%, or by at least 50%. In some embodiments, a stable MAST five-digit score compared to baseline (e.g., prior to treatment or at an earlier time point during treatment) may be indicative of a slowing, delay, or cessation of the progression of SUD, or a reduction in clinical or decline.

The ASI (Addiction Severity Index) is one of the most widely used assessment tools in the United States. It was designed as a research instrument for program evaluation to determine the extent to which addiction treatment programs achieved improvements across seven domains: alcohol use, drug use, psychiatric status, employment status, medical status, legal status, and family/social relationships. Some of these areas have obvious financial implications (e.g., health care utilization, vocational functioning, and arrests). Although frequently used as a primary intake tool, the ASI is best suited for secondary assessment and evaluation after the diagnosis and treatment plan are developed. This instrument is best suited for secondary assessment because the ASI does not provide a basis for a diagnosis, nor does it indicate the urgency for dealing with various conditions.

In some embodiments, a patient that has been treated with rTMS described herein shows an improvement (i.e., an increase) in the patient's ASI score compared to baseline (e.g., prior to treatment or at an earlier time point during treatment). In some embodiments, the patient's ASI score increases by at least 5%, or by at least 10%, or by at least 15%, or by at 20%, or by at least 25%, or by at least 30%, or by at least 35%, or by at least 40%, or by at least 45%, or by at least 50%. In some embodiments, a patient that has been treated with rTMS described herein shows a slowing of the rate of decrease in their ASI score by at least 5%, or by at least 10%, or by at least 15%, at least 20%, or by at least 25%, or by at least 30%, or by at least 35%, or by at least 40%, or by at least 45%, or by at least 50%. In some embodiments, a stable ASI score compared to baseline (e.g., prior to treatment or at an earlier time point during treatment) may be indicative of a slowing, delay, or cessation of the progression of SUD, or a reduction in clinical decline.

In embodiments, e.g. without limitation, in the context of prevention of SUD, the patient is predisposed to addiction. In embodiments, e.g. without limitation, in the context of prevention of SUD, the patient is genetically predisposed to addiction, e.g., without limitation, have a family member who suffers from SUD or addiction. In embodiments, e.g., without limitation, in the context of prevention of SUD, the patient is predisposed to addiction due to having a comorbid disorder, e.g., Bipolar Disorder, Post-Traumatic Stress Disorder (PTSD), and/or Major Depression Disorder (MDD). In embodiments, e.g., without limitation, in the context of prevention of SUD, the patient is a male. In embodiments, e.g., without limitation, in the context of prevention of SUD, the patient is predisposed to addiction due to having low levels of dopamine and/or serotonin.

In embodiments, e.g., without limitation, in the context of conversion of recreational or pharmaceutical drug use to substance use disorder, the patient is predisposed to addiction. In embodiments, e.g., without limitation, in the context of conversion of recreational drug use to substance use disorder, the patient is inclined to use a drug or substance in social contexts and/or for pleasure. In embodiments, e.g., without limitation, in the context of conversion of pharmaceutical drug use to substance use disorder, the patient is prescribed one or more pharmaceutical drugs which are addictive. In embodiments, the patient suffers from chronic pain and/or a debilitating medical condition.

### Additional Therapeutic Agents and Combination Therapy

Administration of rTMS may be combined with additional therapeutic agents. Co-administration of the additional therapeutic agent and the present rTMS may be simultaneous or sequential.

Further, the present methods provide for treatments of patients that are undergoing treatment with one or more In various embodiments, the additional therapeutic agent as described herein.

In one embodiment, the additional therapeutic agent and the rTMS are administered to a subject simultaneously. The term “simultaneously” as used herein, means that the additional therapeutic agent and the rTMS are administered with a time separation of no more than about 60 minutes, such as no more than about 30 minutes, no more than about 20 minutes, no more than about 10 minutes, no more than about 5 minutes, or no more than about 1 minute.

In a further embodiment, the additional therapeutic agent and the rTMS are administered to a subject simultaneously but the release of the additional therapeutic agent from its respective dosage form and the rTMS may occur sequentially.

Co-administration does not require the additional therapeutic agent and the rTMS to be administered simultaneously, if the timing of their administration is such that the pharmacological activities of the additional therapeutic agent and the administration of rTMS overlap in time. For example, the additional therapeutic agent and the rTMS can be administered sequentially. The term “sequentially” as used herein means that the additional therapeutic agent and the rTMS are administered with a time separation of more than about 60 minutes. For example, the time between the sequential administration of the additional therapeutic agent and the administration of rTMS can be more than about 60 minutes, more than about 2 hours, more than about 5 hours, more than about 10 hours, more than about 1 day, more than about 2 days, more than about 3 days, or more than about 1 week apart. The optimal administration times will depend on the rates of metabolism, excretion, and/or the pharmacodynamic activity of the additional therapeutic agent being administered. Either the additional therapeutic agent or the rTMS may be administered first.

In various embodiments, the present invention contemplates rTMS methods used in tandem with one or more additional therapeutic agents. In certain embodiments, the rTMS methods of the present invention obviate the need for treatment with one or more additional therapeutic agents. In some embodiments, the additional therapeutic agent is selected from neurological drugs, opioid antagonists, opioid agonists, cannabinoid receptor antagonist, cannabinoid receptor agonist, mescaline, amphetamine salts, norepinephrine-dopamine reuptake inhibitor, nicotinic receptor antagonist, nicotinic receptor agonists, serotonin reuptake inhibitors, Serotonin-norepinephrine reuptake inhibitors, tricyclic antidepressants, dopamine agonists, dopamine antagonists, typical antipsychotics, atypical antipsychotics, serotonin agonists, serotonin antagonists, Norepinephrine reuptake inhibitors, Monoamine oxidase inhibitors, NMDA receptor antagonists, esketamine, benzodiazepine and baclofen.

In specific embodiments, the additional therapeutic agent is a treatment for SUD, including, but not limited to, Bupropion, naloxone, nicotine, varenicline, Disulfiram, Naltrexone, Acamprosate, Topiramate, Clonidine, Methadone, Buprenorphine, modafinil, escitalopram and amitriptyline. In various embodiments, the rTMS treatment of the present invention is substantially free of adverse effects, optionally selected from epileptic seizures, nausea, and headache.

In embodiments, the additional therapeutic agent is a dopamine active anti-depressant agent. In embodiments, the additional therapeutic agent is one or more of bupropion, aripiprazole, and sertraline. In some embodiments, aripiprazole is not an anti-depressant agent per se. In another embodiment, the SNRT is selected from duloxetine, venlafaxine, nefazodone, and milnacipran. In another embodiment, the dopamine active augmenting agent is one or more of an amphetamine salt, pramipexole, and ropinirole. In another embodiment, the SSRI is selected from citalopram, dapoxetine, s-citalopram, fluoxetine, fluvoxamine, indalpine, paroxetine, and zimelidine.

In some embodiments, the dopamine active anti-depressant agent is one or more of bupropion, aripiprazole, brexpiprazole, and sertraline. Bupropion ((±)-2-(tert-Butylamino)-1-(3-chlorophenyl)propan-1-one), without wishing to be bound by theory, may have its primary pharmacological action through norepinephrine-dopamine reuptake inhibition. It binds selectively to the dopamine transporter, but its behavioral effects may be attributed to its inhibition of norepinephrine reuptake. It also may act as a nicotinic acetylcholine receptor antagonist. Aripiprazole (7-{4-[4-(2,3-Dichlorophenyl)piperazin-1-yl]butoxy}-3,4-dihydroquinolin-2(1H)-one) and brexpiprazole (7-{4-[4-(1-benzothiophen-4-yl)piperazin-1-yl]butoxy}quinolin-2(1H)-one)), without wishing to be bound by theory, are partial dopamine agonist of the second generation class of atypical antipsychotics with additional antidepressant properties that is used in the treatment of schizophrenia, bipolar disorder, and clinical depression. It is approved by the U.S. FDA and EMA for various uses. Without wishing to be bound by theory, Aripiprazole is a dopamimetic at low doses; for example, below 10 mg, or below 9 mg, or below 8 mg, or below 7 mg, or below 6 mg, or below 5 mg, or below 4 mg, or below 3 mg, or below 2 mg, or below 1 mg. Sertraline ((1S,4S)-4-(3,4-dichlorophenyl)-N-methyl-1,2,3,4-tetrahydronaphthalen-1-amine) is a compound of various mood disorder mechanisms. Without wishing to be bound by theory, it may be a dopamine active antidepressant; for example, at doses of 150 mg or above (for example, 160, or 170, or 180, or 190, or 200 mg). Without wishing to be bound by theory, it may also be an antidepressant of the selective serotonin reuptake inhibitor (SSRI) class. It is approved by the U.S. FDA. In some embodiments, the present invention provides methods and compositions comprising doses of sertraline at doses at which sertraline acts as an inhibitor of dopamine uptake. For example, the present invention encompasses doses of sertraline of doses of above 150 mg daily or above 200 mg daily or above 250 mg daily.

In some embodiments, the additional therapeutic agent comprises serotonin-norepinephrine reuptake inhibitors (SNRIs). SNRIs include agents which act upon, and increase, the levels of the neurotransmitters serotonin and norepinephrine, which play an important role in mood. In some embodiments, the SNRI is one or more of duloxetine ((+)-(S)—N-Methyl-3-(naphthalen-1-yloxy)-3-(thiophen-2-yl)propan-1-amine), venlafaxine ((RS)-1-[2-dimethylamino-1-(4-methoxyphenyl)-ethyl]cyclohexanol), nefazodone (1-(3-[4-(3-chlorophenyl)piperazin-1-yl]propyl)-3-ethyl-4-(2-phenoxyethyl)-1H-1,2,4-triazol-5 (4H)-one), and milnacipran ((1R*,2S*)-2-(aminomethyl)-N,N-diethyl-1-phenylcyclopropanecarboxamide). These agents are approved by the U.S. FDA for various uses. For example, the present invention encompasses doses of duloxetine of doses of above 60 mg daily or above 80 mg daily or above 100 mg daily. Further, SNRIs are also provided in certain embodiments, including, for example, desvenlafaxine, tramadol, and sibutramine.

In some embodiments, the additional therapeutic agent comprises dopamine active augmenting agents. In some embodiments dopamine active augmenting agents include agents that may be used in the treatment of SUD or co morbid psychiatric symptoms in patients with SUD. In some embodiments, the dopamine active augmenting agent is one or more of an amphetamine salt, pramipexole, and ropinirole. Amphetamine salts include, for example, ADDERALL, a combination of four amphetamine salts (racemic amphetamine aspartate monohydrate, racemic amphetamine sulfate, dextroamphetamine saccharide, and dextroamphetamine sulfate). ADDERALL, without wishing to be bound by theory, is a dopamine releasing agent, a norepinephrine releasing agent, and can be mildly serotonergic. Pramipexole ((S)—N⁶-propyl-4,5,6,7-tetrahydro-1,3-benzothiazole-2,6-diamine)), without wishing to be bound by theory, is a dopamine agonist of the non-ergoline class. Ropinirole (4-[2-(dipropylamino)ethyl]-1,3-dihydro-2H-indol-2-one)), without wishing to be bound by theory, is also a dopamine agonist of the non-ergoline class of medications.

In some embodiments, the additional therapeutic agent comprises selective serotonin re-uptake inhibitors (SSRIs). SSRIs include agents which act upon, and increase, the levels of the neurotransmitter serotonin, which plays an important role in mood. In some embodiments, the SSRI is one or more of citalopram ((RS)-1-[3-(dimethylamino)propyl]-1-(4-fluorophenyl)-1,3-dihydroisobenzofuran-5-carbonitrile), Dapoxetine(S)—N,N-dimethyl-3-(naphthalen-1-yloxy)-1-phenylpropan-1-amine), S-Citalopram ((S)-1-[3-(dimethylamino)propyl]-1-(4-fluorophenyl)-1,3-dihydroisobenzofuran-5-carbonitrile), Fluoxetine ((RS)—N-methyl-3-phenyl-3-[4-(trifluoromethyl)phenoxy] propan-1-amine), Fluvoxamine ((E)-5-methoxy-1-[4-(trifluoromethyl)phenyl]pentan-1-one O-2-aminoethyl oxime), Indalpine (3-(2-piperidin-4-ylethyl)-1H-indole), Paroxetine ((3S,4R)-3-[(2H-1,3-benzodioxol-5- yloxy)methyl]-4-(4-fluorophenyl)piperidine), and Zimelidine ((Z)-3-(4-bromophenyl)-N,N-dimethyl-3-(pyridin-3-yl)prop-2-en-1-amine). Further SSRIs are also provided in certain embodiments, including, for example, citalopram, escitalopram, paroxetine, fluoxetine, and fluvoxamine.

In various embodiments, the additional therapeutic agent comprises serotonin antagonist and reuptake inhibitors (SARIs), such as, for example, etoperidone, lubazodone, nefazodone, and trazodone.

In various embodiments, the additional therapeutic agent comprises norepinephrine reuptake inhibitors (NRIs), such as, for example, atomoxetine, reboxetine, and viloxazine. In still other embodiments, further agents may include norepinephrine-dopamine reuptake inhibitors (NDRIs), such as, for example, bupropion, dexmethylphenidate, methylphenidate, and methylphenidate.

In various embodiments, the additional therapeutic agent comprises norepinephrine-dopamine releasing agents (NDRAs), such as, for example, amphetamine, various amphetamine salts (e.g. salts of racemic amphetamine and dextroamphetamine, Adderall), dextroamphetamine, dextromethamphetamine, lysine-amphetamine (e.g. Vyvanase) and lisdexamfetamine.

In various embodiments, the additional therapeutic agent comprises tricyclic antidepressants (TCAs), such as, for example, amitriptyline, butriptyline, clomipramine, desipramine, dosulepin, doxepin, imipramine, iprindole, lofepramine, melitracen, nortriptyline, opipramol, protriptyline, and trimipramine.

In various embodiments, the additional therapeutic agent comprises tetracyclic antidepressants (TeCAs), such as, for example, amoxapine, maprotiline, mianserin, and mirtazapine.

In various embodiments, the additional therapeutic agent comprises monoamine oxidase inhibitors (MAOIs), such as, for example, isocarboxazid, moclobemide, phenelzine, pirlindole, selegiline, and tranylcypromine.

In embodiments the additional therapeutic agent is a nicotine replacement therapy. In embodiments the additional therapeutic agent is a nicotine patch, gum, lozenge, and/or inhaler e.g. NICODERM, NICORETTE, and the like. In embodiments the additional therapeutic agent is varenicline, e.g. CHANTIX and/or bupropion, e.g. ZYBAN.

In embodiments the additional therapeutic agent is naltrexone, e.g., without limitation, for embodiments when the substance is alcohol.

In embodiments the additional therapeutic agent is methadone, e.g. DOLOPHINE and METHADOSE. In embodiments the additional therapeutic agent is suboxone, e.g. DOLOPHINE and METHADOSE.

In embodiments the additional therapeutic agent is buprenorphine. In embodiments the additional therapeutic agent is naloxone. In embodiments the additional therapeutic agent is a combination buprenorphine and naloxone, e.g. SUBOXONE or ZUBSOLV.

In embodiments the substance is an opioid, e.g. without limitation, in the context of opioid use disorder, and the additional therapeutic agent is methadone, e.g. DOLOPHINE and METHADOSE. In embodiments the substance is an opioid, e.g. without limitation, in the context of opioid use disorder, and the additional therapeutic agent is suboxone, e.g. DOLOPHINE and METHADOSE. In embodiments the substance is an opioid, e.g. without limitation, in the context of opioid use disorder, and the additional therapeutic agent is buprenorphine. In embodiments the substance is an opioid, e.g. without limitation, in the context of opioid use disorder, and the additional therapeutic agent is naloxone. In embodiments the substance is an opioid, e.g. without limitation, in the context of opioid use disorder, and the additional therapeutic agent is a combination buprenorphine and naloxone, e.g. SUBOXONE or ZUBSOLV.

### Definitions

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50%” covers the range of 45% to 55%.

An “effective amount,” when used in connection with medical uses is an amount that is effective for providing a measurable treatment, prevention, or reduction in the rate of pathogenesis of a disorder of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of a stimulus, relative to the absence of such stimulus.

As used herein, the word “include,” and its variants, is intended to be non-limiting, such that recitation of items in a list is not to the exclusion of other like items that may also be useful in the treatments and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

An effective amount of the treatment as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein.

### EXAMPLES

### Example 1: High Frequency and Low Intensity Transcranial Magnetic Stimulation for Smoking Cessation

The objective of this study was to evaluate the smoking cessation rate (point and continuous) after 8 weeks of treatment with HFLI TMS in smokers with moderate-to-severe nicotine dependence; CO exhaled concentration was used as an objective measurement to determine efficacy of intervention and as the main outcome of this study.

Health records from a private neuromodulation clinic were screened for ambulatory smoker patients that received at least 24 sessions of HFLI TMS for smoking cessation, without skipping any sessions. Subjects had to smoke at least ten cigarettes per day continuously during one year before the intervention. Patients were not considered for this case series if they had a previous history of substance abuse other than tobacco, chronic diseases out of medical control, morbid obesity, clinically diagnosed mental disorder, and/or epilepsy. Subjects had to have at least 2 months without any active intervention to stop smoking before starting HFLI TMS; subjects that received some type of counseling during the treatment duration were not considered for this study. All clinical data and assessments were retrieved retrospectively from the health records of the clinic. After gathering the records, all patients that completed the previous requirements were contacted and all signed a written informed consent and agreed to the publication of their cases; because of the retrospective nature of the study and data recollection characteristics, no ethics committee approval was required by the institution.

In all reported patients, stimulation was applied every third day for 8 weeks with a total of 24 sessions. In each session, HFLI TMS was applied with a circular coil with 60 mm diameter over the left dorsolateral prefrontal cortex. To stimulate this area, the 10-20 EEG coordinates of F3 were measured in the skull of each participant; the center of the coil was located in this spot. Stimulation protocol consisted of 1 continuous train at 575 Hz for 45 minutes with each pulse intensity at around 0.5 milliteslas. During the sessions, patients sat in a comfortable chair and were allowed to move freely without standing up.

Clinical history, epidemiological data, Fagerström Test for Nicotine Dependence (FTDN), Test to Assess the Psychological Dependence on Smoking (TAPDS), and Alcohol Use Disorders Identification Test (AUDIT) were acquired or measured before starting the intervention. The Beck Depression Inventory (BDI), Beck Anxiety Inventory (BAI), and the Short Form (SF36) Health Survey were measured before starting the first stimulation session and after the last stimulation session. See FIG. 5. To evaluate smoking cessation, subjects performed a weekly determination of exhaled carbon monoxide (CO) with a carbon monoxide analyzer using as cutoff points values below 10 ppm to determine smoking cessation; the same test and criteria were applied to measure continuous abstinence after 3 months of the intervention. Also, patients registered in a diary if they smoked any cigarette during the 8 weeks of stimulation to determine point abstinence and continuous abstinence. The possible adverse effects were assessed after stimulation in each session. Weight and blood pressure were measured weekly. All these assessments, including a copy of the smoking dairy, were retrieved from the clinical record of each patient.

Our sample was collected by convenience as only ten different patients matched the previously defined criteria from a total pool of 50 patients that had received HFLI TMS for smoking cessation and other indications in the private clinic. All subjects attended stimulation every third day for 8 weeks receiving a total of 24 sessions. Clinical and epidemiological data of the ten patients are summarized in Table A:

### Clinical and Epidemiological Data of Reported Subjects

| | | Subject | Subject | Subject | Subject | Subject | Subject | Subject | Subject | Subject | Subject | Mean |
|---|---|---|---|---|---|---|---|---|---|---|---|---|
| | | 1 | 2 | 3 | 4 | 5 | 6 | 7 | 8 | 9 | 10 | (SD) |
| | Gender | M | M | F | M | F | F | F | F | M | M | |
| | Age (years) | 57.00 | 64.00 | 65.00 | 39.00 | 59.00 | 55.00 | 50.00 | 61.00 | 46.00 | 72.00 | 56.80 |
| | | | | | | | | | | | | (9.75) |
| | Years smoked | 27.00 | 46.00 | 48.00 | 21.00 | 39.00 | 37.00 | 20.00 | 43.00 | 31.00 | 57.00 | 36.90 |
| | | | | | | | | | | | | (12.12) |
| | Cigarettes/day | 30.00 | 10.00 | 15.00 | 20.00 | 8.00 | 20.00 | 40.00 | 10.00 | 20.00 | 18.00 | 19.10 |
| | | | | | | | | | | | | (9.80) |
| | Packets/year | 40.50 | 23.00 | 36.00 | 21.00 | 15.60 | 37.00 | 40.00 | 21.50 | 31.00 | 51.30 | 31.69 |
| | | | | | | | | | | | | (11.21) |
| | Attempts to | 1.00 | 0 | 3.00 | 2.00 | 3.00 | 2.00 | 1.00 | 2.00 | 1.00 | 5.00 | 2.00 |
| | quit smoking | | | | | | | | | | | (1.41) |
| | BMI | 30.01 | 23.18 | 36.07 | 26.89 | 25.78 | 23.13 | 23.08 | 25.02 | 29.13 | 25.14 | 26.74 |
| | | | | | | | | | | | | (4.07) |
| | FTND score | 9.00 | 2.00 | 7.00 | 3.00 | 1.00 | 7.00 | 9.00 | 3.00 | 7.00 | 8.00 | 5.60 |
| | | | | | | | | | | | | (3.03) |
| | TAPD score | 59.00 | 32.00 | 53.00 | 39.00 | 39.00 | 48.00 | 59.00 | 52.00 | 63.00 | 58.00 | 50.20 |
| | | | | | | | | | | | | (10.42) |
| | AUDIT score | 4.00 | 0 | 2.00 | 1.00 | 1.00 | 1.00 | 2.00 | 0 | 5.00 | 0 | 1.60 |
| | | | | | | | | | | | | (1.71) |
| BDI | Baseline | 20.00 | 1.00 | 15.00 | 4.00 | 10.00 | 27.00 | 19.00 | 12.00 | 17.00 | 4.00 | 12.9 |
| | | | | | | | | | | | | (8.28) |
| | Post-HFLI | 22.00 | 0 | 9.00 | 0 | 7.00 | 18.00 | 32.00 | 2.00 | 1.00 | 7.00 | 9.80 |
| | TMS | | | | | | | | | | | (10.83) |
| BAI | Baseline | 9.00 | 0 | 15.00 | 0 | 1.00 | 23.00 | 21.00 | 18.00 | 2.00 | 25.00 | 11.40 |
| | | | | | | | | | | | | (10.17) |
| | Post-HFLI | 8.00 | 1.00 | 7.00 | 0 | 6.00 | 16.00 | 27.00 | 0 | 0 | 2.00 | 6.70 |
| | TMS | | | | | | | | | | | (8.76) |
| SF36% | Baseline | 64.75 | 88.52 | 90.16 | 98.36 | 95.08 | 82.79 | 90.16 | 92.62 | 93.44 | 68.03 | 86.39 |
| | | | | | | | | | | | | (11.35) |
| | Post-HFLI | 84.43 | 86.07 | 98.36 | 93.44 | 90.16 | 98.36 | 95.08 | 87.70 | 91.80 | 88.52 | 91.39 |
| | TMS | | | | | | | | | | | (4.89) |
| CO | Baseline | 30 | 5 | 22 | 34 | 9 | 50 | 19 | 2 | 10 | 22 | 20.3 |
| ppm | | | | | | | | | | | | (14.81) |
| | Post-HFLI | 0 | 0 | 0 | 1 | 0 | 0 | 8 | 0 | 3 | 1 | 1.3 |
| | TMS | | | | | | | | | | | (2.54) |

The mean age was 56.80 years (SD±9.75); on average, subjects smoked 19.10 cigarettes each day (range 8-40) for 36.90 years (range 20-57) and had attempted 2 times (range 0-5) to quit smoking. We found a moderate-to-very high dependence on nicotine assessed by the FTND score (range 1-9), an AUDIT score of 1.6 (range 0-5), and a moderate-to-high psychological dependence with TAPDS (range 32-63). After the 24 sessions, all patients stopped smoking; this was confirmed directly by exhaled carbon monoxide and the smoking diary, thereby showing achievement of point abstinence. Three months after the intervention was finished, eight out of ten subjects continued without smoking according to personal reports and CO measures, thus showing continuous abstinence. No severe adverse effects were reported by participants. Participants were compliant to each session without reprogramming.

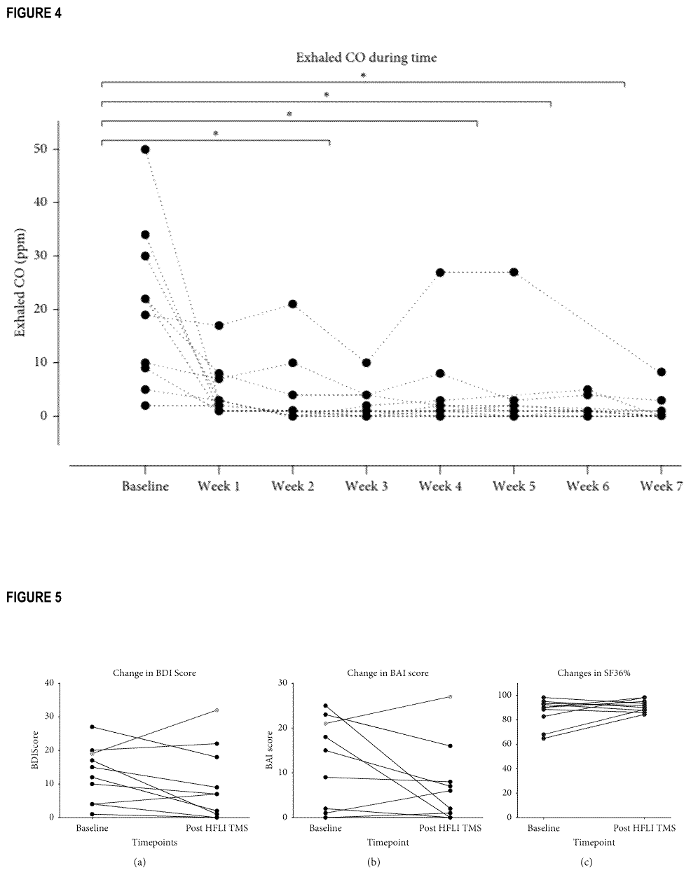

To evaluate changes of exhaled CO concentrations along time, a Friedman test was conducted. Exhaled CO concentrations were statistically significantly different at the evaluated time points, χ2(7)=32.409, <0.01. Post hoc pairwise comparisons were performed with a Bonferroni correction for multiple comparisons; statistical significance was accepted at the <0.01 level. A statistically significant difference was observed in CO concentration from baseline to week 3 concentration (<0.01), week 5 concentration (<0.01), week 6 concentration (<0.01), and last measured concentration (<0.01). See FIG. 4.

Results show that after 24 sessions of HFLI magnetic fields, all patients stopped smoking; this was confirmed directly by exhaled carbon monoxide and the smoking diary. See FIG. 4. Three months after intervention, eight out of ten subjects continued without smoking.

### Equivalents

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### Incorporation by Reference

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

### REFERENCES

American Psychiatric Association, & American Psychiatric Association. (2013). Diagnostic and statistical manual of mental disorders: DSM-5. Arlington, VA
McCabe, S. E., West, B. T., Jutkiewicz, E. M., & Boyd, C. J. (2017). Multiple DSM-5 substance use disorders: A national study of US adults. Human Psychopharmacology: Clinical and Experimental, 32(5), e2625.
United Nations. (2019). World Drug Report 2019:35 million people worldwide suffer from drug use disorders while only 1 in 7 people receive treatment. On World Wide Web at unodc.org/unodc/index.html
Wu, L. T., McNeely, J., Subramaniam, G. A., Brady, K. T., Sharma, G., VanVeldhuisen, P., & Schwartz, R. P. (2017). DSM-5 substance use disorders among adult primary care patients: Results from a multisite study. Drug and alcohol dependence, 179, 42-46.

What is claimed is:

1. A repetitive transcranial magnetic stimulation (rTMS) method for treating or preventing substance use disorder (SUD) or slowing or preventing a conversion of recreational or pharmaceutical drug use to substance use disorder, comprising repetitively applying a magnetic pulse to the scalp of a patient in need thereof thereby stimulating neurons in the brain of the patient, wherein the magnetic pulse is applied:
repetitively over the patient's brain; and
at a frequency of about 10 to about 1000Hz and an intensity of about 5,000 to about 150,000 milligauss.

2. A rTMS method for slowing or preventing a conversion of recreational or pharmaceutical drug use to substance use disorder, comprising repetitively applying a magnetic pulse to the scalp of a patient in need thereof thereby stimulating neurons in the brain of the patient, wherein the magnetic pulse is applied:
repetitively over the patient's brain; and
at a frequency of about 10 to about 1000 Hz and an intensity of about 5,000 to about 150,000 milligauss.

3. The method of either claim 1 or 2, wherein the magnetic pulse is applied at a frequency of about 400 to about 600 Hz.

4. The method of claim 3, wherein the magnetic pulse is applied at a frequency of about 575 Hz.

5. The method of any one of the previous claims, wherein the magnetic pulse is applied at an intensity of about 10,000milligauss.

6. The method of any one of the previous claims, wherein the magnetic pulse generates an electric field of about 0.1 to about 10 V/m⁷.

7. The method of claim 6, wherein the magnetic pulse generates an electric field of about 0.5 to about 1.5 V/m⁷.

8. The method of claim 7, wherein the magnetic pulse generates an electric field of about 1 V/m⁷.

9. The method of any one of the previous claims, wherein the method is undertaken once, or twice, or thrice, or four times daily.

10. The method of claim 9, wherein the method is undertaken twice daily.

11. The method of any one of the previous claims, wherein the method is undertaken for greater than about 15 minutes.

12. The method of any one of the previous claims, wherein the method is undertaken for about 15 to about 60 minutes.

13. The method of claim 12, wherein the method is undertaken for about 30 minutes.

14. The method of any one of the previous claims, wherein the pulses are applied about 300 to about 400 times.

15. The method of claim 14, wherein the pulses are applied about 360 times.

16. The method of any one of the previous claims, wherein the pulses are applied discontinuously.

17. The method of any one of the previous claims, wherein the pulses last for about three seconds.

18. The method of claim 17, wherein the pulses last for about three seconds and followed by one second without pulsing.

19. The method of any one of the previous claims, wherein the treatment is applied chronically.

20. The method of any one of the previous claims, wherein the treatment is applied for greater than about 2 months.

21. The method of claim 20, wherein the treatment is applied for greater than about 6 months.

22. The method of claim 21, wherein the treatment is applied for greater than about one year.

23. The method of any one of the previous claims, wherein the treatment is self-applied.

24. The method of any one of the previous claims, wherein the magnetic pulse is applied using a device, the device being suitable for conducting electric current through a coil.

25. The method of claim 24, wherein the device generates a magnetic field.

26. The method of claim 25, wherein the device is suitable for home use.

27. The method of claim 26, wherein the device is portable.

28. The method of any one of the previous claims, where in the patient is afflicted with SUD.

29. The method of any one of the previous claims, wherein the patient is afflicted with substance specific induced disorders such as intoxication, withdrawal or substance induced mental disorders caused by alcohol, caffeine, cannabis, hallucinogens, inhalants, opioids, sedatives, hypnotics, anxiolytics, stimulants, cocaine, MDMA, nicotine, and/or tobacco.

30. The method of any one of the preceding claims, wherein the patient presents as having the diagnosis of SUD according to Diagnostic and Statistical Manual of Mental Disorders (DSM) V.

31. The method of any one of the preceding claims, wherein the patient presents as having a test score of one or more identified instruments for assessment of SUD or SUD related psychiatric symptoms that is considered to indicate the presence of SUD or SUD related psychiatric symptoms, optionally selected from Table 1.

32. The method of any one of the preceding claims, wherein the method provides an improvement in a test score of one or more identified instruments for assessment of SUD or SUD related psychiatric symptoms that is considered to indicate the presence of SUD or SUD related psychiatric symptoms, optionally selected from Table 1.

33. The method of any one of the preceding claims, wherein the method stimulates neurons around about 2 to about 3 cm from the skull.

34. The method of any one of the preceding claims, wherein the magnetic pulse is applied repetitively over the patient's left prefrontal dorsolateral cortex.

35. The method of any one of the preceding claims, wherein the method stimulates neurons throughout the patient's brain.

36. The method of any one of the preceding claims, wherein the method substantially stimulates neurons in the left hemisphere of the patient's brain and/or frontal lobe of the patient's brain.

37. The method of any one of the preceding claims, wherein the method substantially stimulates neurons in the cerebral cortex of the patient.

38. The method of any one of the preceding claims, wherein the method stimulates neurons outside of the patient's left prefrontal dorsolateral cortex.

39. The method of any one of the preceding claims, wherein the method prevents or delays the conversion of recreational or pharmaceutical drug use to substance use disorder.

40. The method of any one of the preceding claims, wherein the treatment improves SUD symptoms of the patient.

41. The method of any one of the preceding claims, wherein the treatment curtails or prevents exacerbation of SUD.

42. The method of any one of the preceding claims, wherein the treatment is used in tandem with one or more additional therapeutic agents.

43. The method of any one of the preceding claims, wherein the treatment obviates the need for treatment with one or more additional therapeutic agents.

44. The method of any one of the preceding claims, wherein the treatment is substantially free of adverse effects, optionally selected from epileptic seizures, nausea, and headache.
